(19) United States
(12) Patent Application Publication
(10) Pub. No.: US 2024/0316285 A1
(43) Pub. Date: Sep. 26, 2024

(54) DISTRACTION DEVICE FOR NEEDLE INSERTION INTO TISSUE

(71) Applicant: Prudenfort Pty Ltd, Bondi Junction, New South Wales (AU)

(72) Inventor: Dave Roberto Listijono, Bondi Junction (AU)

(21) Appl. No.: 18/575,149

(22) PCT Filed: Jun. 27, 2022
(86) PCT No.: PCT/AU2022/050658

§ 371 Date: Dec. 28, 2023

(30) Foreign Application Priority Data
Jun. 30, 2021 (AU) 2021204566

(51) Int. Cl. A61M 5/42; A61M 5/158; A61M 5/32

(52) U.S. Cl. CPC A61M 5/425; A61M 5/3287; A61M 2005/1585

(57) ABSTRACT

A distraction device configured to be used with a needle device for insertion into tissue includes a body attached to the needle device; and a member extending from the body, wherein the member includes a distractor tip proximal to a needle tip of the needle device. In use, the distractor tip is configured to contact a tissue surface before and during insertion of the needle tip into the tissue surface.

### BACKGROUND

### Technical Field

Aspects of the disclosure relate generally to a needle device, and a distractor device used with needles for medical use. The present disclosure may be used during insertion of a cannula.

### Description of the Related Art

The use of needles for medical purposes can cause physical and psychological discomfort. A needle piercing the skin can cause acute pain for a patient. Furthermore, the anticipation of such pain can also cause mental discomfort. In some cases, patients can have a fear of needles that can lead to reluctance to accept testing or treatment that involves needles or other sharp medical devices.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### BRIEF SUMMARY

There is provided a distraction device configured to be used with a needle device for insertion into tissue, comprising: a body attached to the needle device; a member extending from the body, the member comprising a distractor tip proximal to a needle tip of the needle device; wherein in use the distractor tip is configured to contact a tissue surface before and during insertion of the needle tip into the tissue surface.

The distractor tip can provide a touch sensation to the patient to distract their sensation from insertion of the needle tip. In particular, the distractor tip can provide a mild prick sensation that is not painful to the patient. This prick sensation can distract the patient before the needle tip penetrates the tissue surface. Since the site of the mild prick sensation is close to the site of the needle tip penetration the tissue surface, the distractor tip can reduce the pain sensation of needle insertion.

In the distraction device, the body may be removably attached to the needle device. In the distraction device, the body may comprise an adapter to interface the body with the needle device.

The adapter may comprise a slotted tube to slidingly receive the needle device.

In the distraction device, the member may be removably attached to the body. The member may be resiliently flexible such that the member is configured to bend when the distractor tip is in contact with the tissue surface during insertion of the needle tip into the tissue surface. The resiliently flexible member may be configured to apply pressure on the tissue surface without causing penetration. The resiliently flexible member may allow the distractor tip to remain at a same location on the tissue surface during insertion of the needle tip.

In the distraction device, the distractor tip may be located ahead of the needle tip of the needle device. That is, during insertion the distractor tip contacts the tissue surface first and subsequent movement along a needle axis causes the needle tip to contact the tissue surface at, or proximal to the point of the distractor tip. In some examples, the distractor tip may be located adjacent to the needle tip of the needle device. This can include the distractor tip located close, but slightly off-axis to the axis of the needle.

The distraction device may further comprise a stabilizer grip to stabilize the distraction device before and during insertion of the needle tip into the tissue surface. The stabilizer grip may comprise a pair of resilient flaps that are selectively attached to the needle device. The pair of resilient flaps may fold towards the body such that digits of an operator of the needle device can squeeze the stabilizer grip with part of the body between.

A needle device for insertion into tissue, comprising: a body; a resiliently flexible member extending from the body of the needle device, wherein the resiliently flexible member includes a distractor tip; and a needle with a needle tip, wherein the distractor tip is proximal to the needle tip, wherein in use the distractor tip is configured to contact a tissue surface of a patient before and during insertion of the needle tip into the tissue surface. In the needle device, the resiliently flexible member may be configured to bend during insertion of the needle tip into the tissue surface.

In the needle device, the resiliently flexible member may be configured to apply pressure to the tissue surface without causing penetrations.

In some examples of the needle device, the resiliently flexible member allows the distractor tip to remain at a same location on the tissue surface during insertion of the needle tip.

In some examples of the needle device, the distractor tip is located ahead of the needle tip.

In some examples of the needle device, the distractor tip is located adjacent to the needle tip of the needle device.

### BRIEF DESCRIPTION OF THE SEVERAL VIEWS OF THE DRAWINGS

Examples of the present disclosure will be described with reference to:

FIG. 1 illustrates a schematic diagram of an example distraction device;

FIG. 2 illustrates a top view of an example distraction device;

FIG. 3 illustrates a side view of an example distraction device;

FIG. 4 illustrates an underside view of an example distraction device;

FIG. 5 illustrates a front view of an example distraction device;

FIG. 6 illustrate a rear view of an example distraction device;

FIG. 7 illustrates an exploded view of an example distraction device;

FIGS. 8A and 8B illustrate an example adaptor;

FIGS. 9A and 9B illustrate an example body;

FIGS. 10A and 10B illustrate an example schematic distractor tip;

FIG. 11 illustrates an example distraction device; and

FIG. 12 illustrates an exploded view of an example distraction device.

### DETAILED DESCRIPTION

### Overview

A distraction device configured to be used with a needle device for insertion into tissue will now be described.

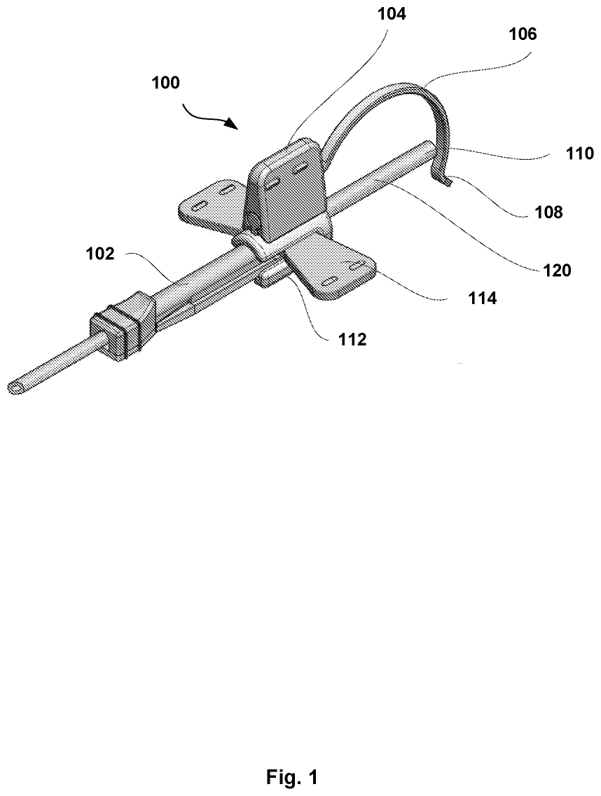

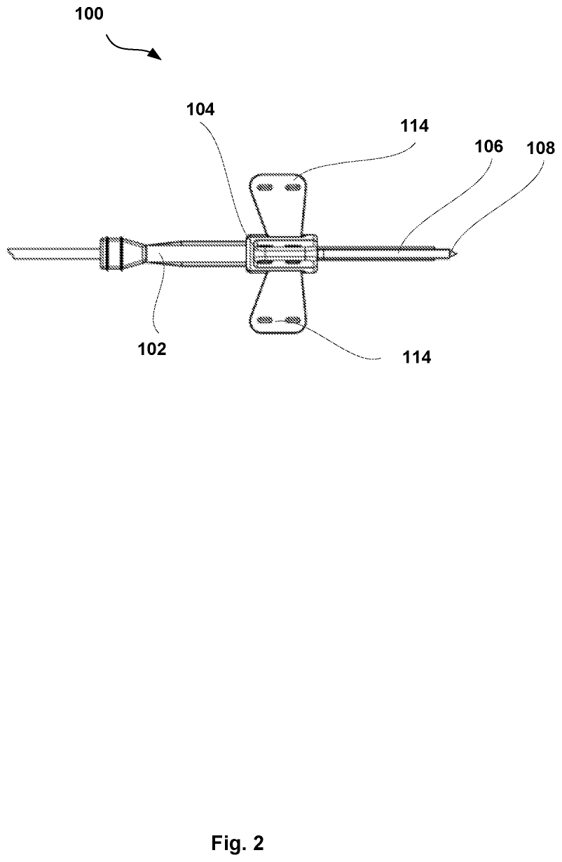

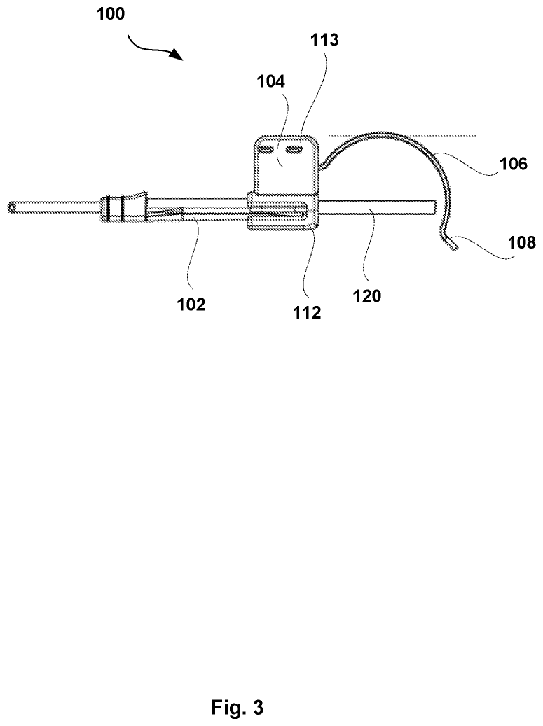

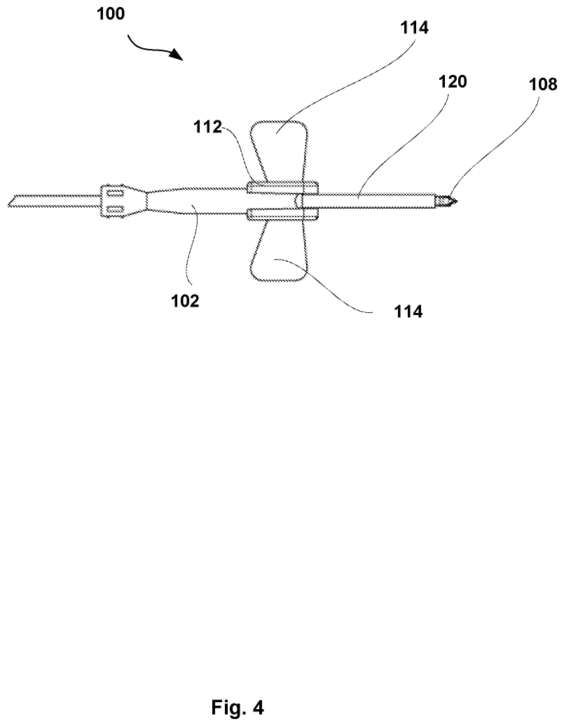

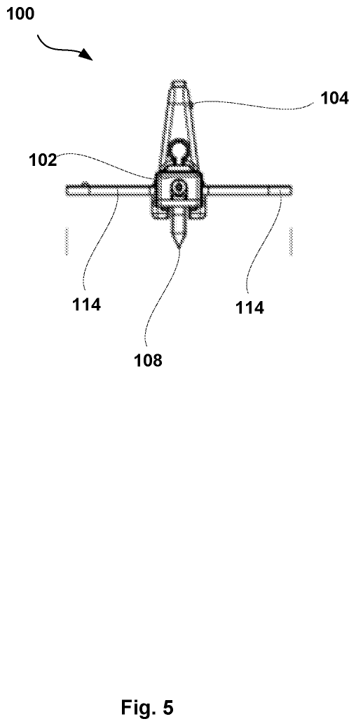

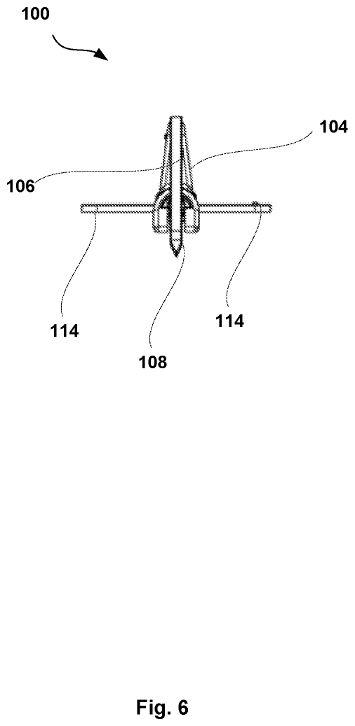

FIGS. 1 to 7 illustrates a distraction device 100 that is configured to be used with a needle device 102 for insertion into tissue. Examples of a needle device may comprise venipuncture devices such as syringes, butterfly needles, catheters, and infusion needles. The distraction device 100 comprises a body 104 attached to the needle device 102, and a member 106 extending from the body 104. The member 106 comprises a distractor tip 108 proximal to a needle tip 110 of the needle device 103. In some examples the distractor tip 108 may be located adjacent to the needle tip 110 of the needle device 103.

In use, the distractor tip 108 of the distraction device 100 is configured to contact a tissue surface before and during insertion of the needle tip 110 into the tissue surface. In some examples the tissue surface may comprise a skin surface of a human or animal.

Details of an exemplary distraction device 100 and needle device 102 will now be described in detail.

### Body 104

As described above the distraction device 100 comprises a body 104 attached to the needle device 102. In some examples, the body 104 may be removably attached to the needle device 102. In a further example, the body 104 may be slidably received by the needle device 102. In this way the body 104 may comprise a cavity to receive the needle device 102.

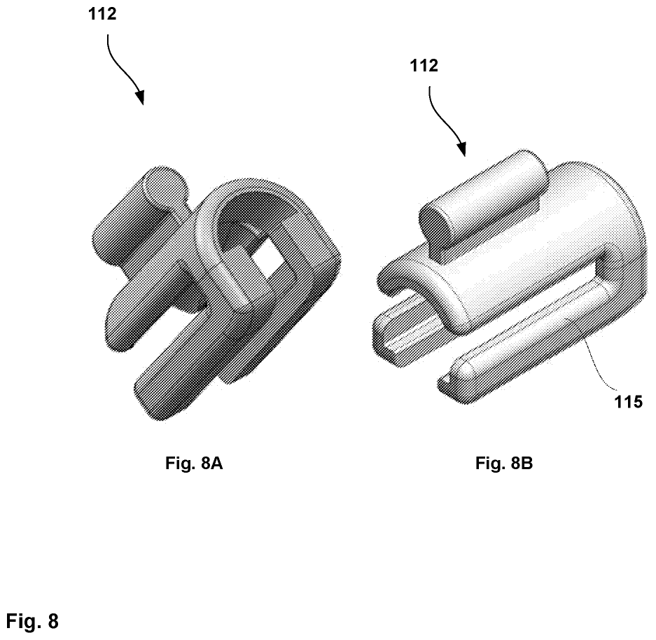

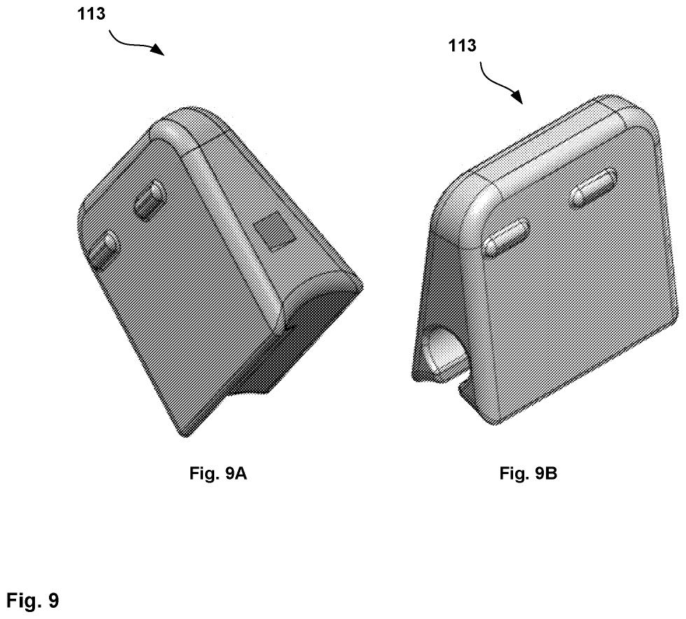

In another example, the body 104 may two portions, a main body portion 113 and an adapter 112 to interface the body 104 with the needle device 102. This is illustrated in FIGS. 8A to 9B where the body 104 includes the main body portion 113 and adapter 112. In this example, the adapter 112 may comprise a slotted tube 115 to slidingly receive the needle device 102. In another example, the adapter 112 may attach to the needle device 102 such that the body 104 is securely interfaced with the needle device 102. For example, the adapter 112 may fasten to the needle device 102 via a locking mechanism.

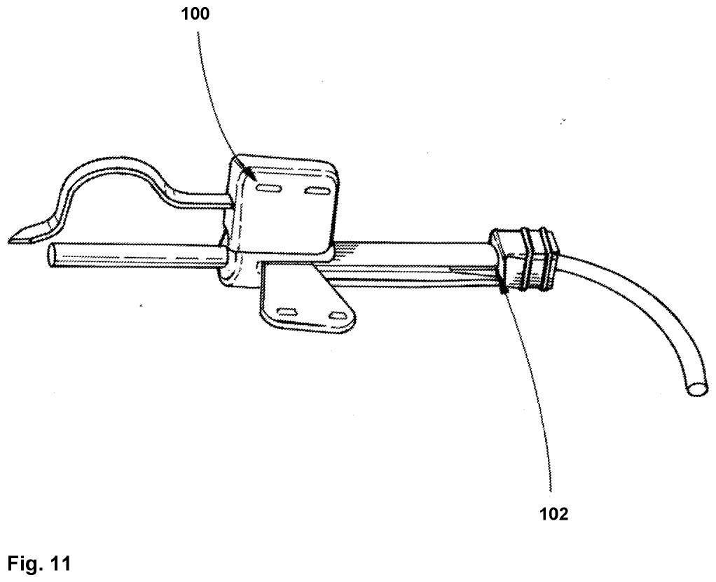

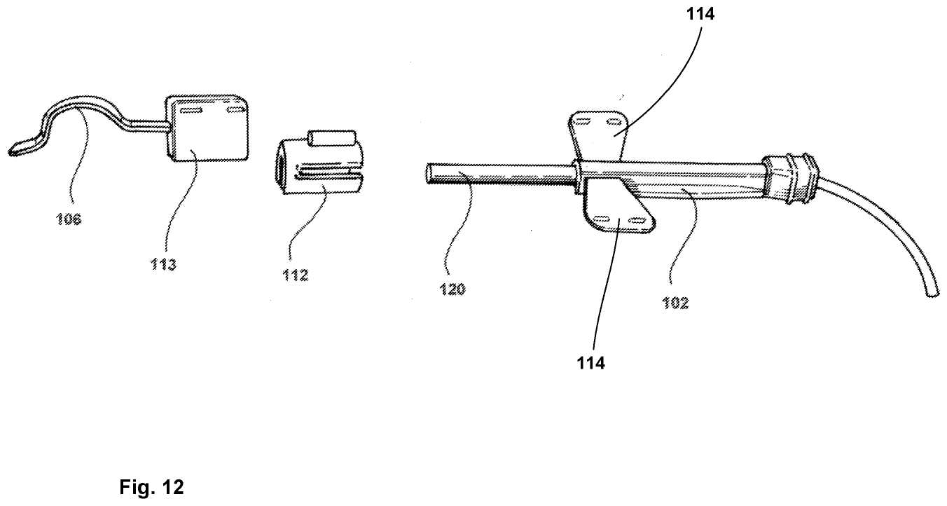

An advantage of utilizing an adapter 112 is to enable the main body portion 113 to be adapted for use with different types and sizes of needle device 102. Various adapters 112 can be manufactured for specified needle devices, and the adapters 112 can enable a common main body portion 113 and member 106 to be utilized for various different needle devices. This is best illustrated in FIG. 12 where the distraction device 100 is separated from a needle device 102, where the needle device 102 may be a commercial of the shelf product. By selecting an appropriate adaptor 112, this can be assembled with the main body portion 113 to form a distraction device 100 assembled with the needle device 102 as illustrated in FIG. 11.

### Member 106

As described above, the distraction device 100 comprises a member 106 extending from the body 104. The member comprises a distractor tip 108 proximal to a needle tip 110 of the needle device. In some examples, the member 106 may be removably attached to the body 104.

The member 106 may be resiliently flexible such that the member 106 is configured to bend during insertion of the needle tip 110 into the tissue surface. In this way, the member 106 is able to deform or buckle while the needle tip 110 is inserted into the tissue surface. In some examples, the resiliently flexible member 106 is configured to apply pressure on the tissue surface without causing penetration. That is the resiliently flexible nature of the member 106 maintains pressure on the skin without causing penetration.

The resiliently flexible nature of the member 106 also allows the distractor tip 108 to remain at the same location on the skin during insertion of the needle tip 110 into the tissue surface.

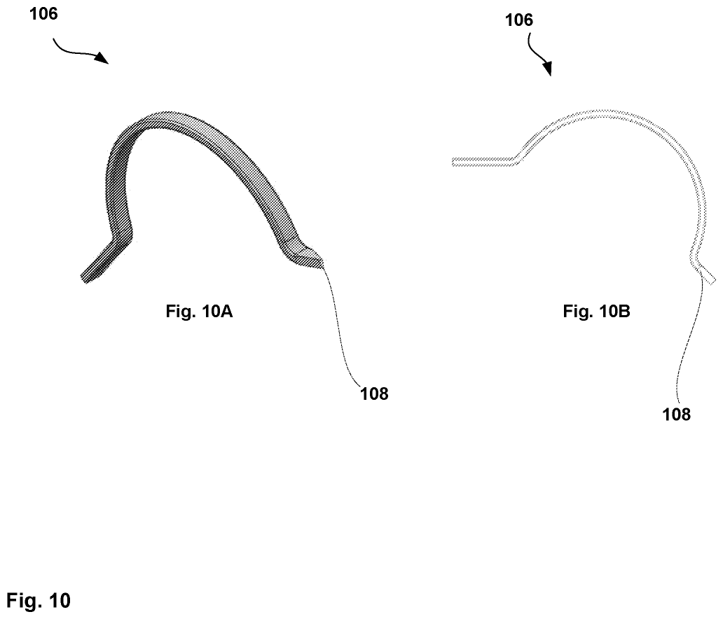

In some examples, the member 106 may be a curved shape as illustrated in FIGS. 1, 10A, and 10B. This can assist in controlled deformation and buckling, and controlled application of pressure at the distractor tip 108. In other examples, the member 106 may be a substantially linear shape.

### Distractor Tip 108 and Needle Tip 110

As also described above, the member 106 of the distraction device 100 comprises a distractor tip 108 proximal to a needle tip 110 of the needle device 102. In use, the distractor tip 108 is configured to contact a tissue surface before and during insertion of the needle tip 110 into the tissue surface. In this way, the distractor tip 108 of the member 106 maintains pressure on the tissue surface without causing penetration.

The resiliently flexible member 106 allows the distractor tip 108 to remain at a same location on the tissue surface. This can allow the doctor, nurse, or paramedic to slightly vary an insertion angle or move the needle during insertion while ensuring the distractor tip 108 is in contact with the tissue surface to maintain the distraction effect.

As also described above, the distractor tip 108 is located proximal to the needle tip 110. In one example the distractor tip 108 is located ahead of the needle tip 110 of the needle device 103. This can include locating the distractor tip 108 at a location on the needle axis and in front of the needle tip 110. During insertion of the needle, the distractor tip 108 contacts the tissue surface and creates a mild distraction sensation at a point on the tissue surface. Upon further movement of the needle, the needle tip 110 pierces the needle tip 100 at or proximal to the point on the tissue surface. In some examples, the needle tip 110 follows behind the distractor tip 108, and movement of the needle brushes the distractor tip 108 slightly to the side to enable insertion of the needle tip 110.

In other examples, the distractor tip is located adjacent to the needle tip 110. This may comprise the distractor tip 108 being off-axis to the axis of the insertion of the needle tip 110 into the tissue surface. That is, the distractor tip 108 is configured to provide a mild distraction sensation at a point slightly offset from a penetration point of the needle tip 110 at the tissue surface.

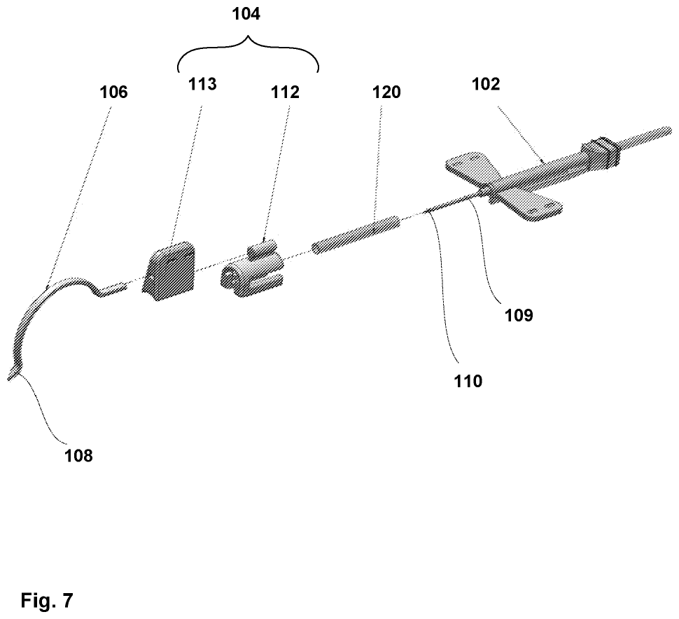

In some examples, the needle 109 may be shielded with a needle sheath 120 for safe storage and protection before use (as illustrated in FIG. 1). The needle sheath 120 is removed before use. The sheath 120 separated from the needle 109 is best illustrated in FIG. 7.

### Variations

### Stabilizer Grips

In some examples, the distraction device 100 further comprises a stabilizer grip 114 to stabilize the distraction device 100 before and during insertion of the needle tip 110 into the tissue surface.

In some examples, the stabilizer grip 114 comprises a pair of resilient flaps that are selectively attached to the needle device. The pair of resilient flaps may fold towards the body 104. In this way, the digits of an operator of the needle device 100 can squeeze the stabilizer grip with part of the body 104 between.

Needle Device with Resilient Flexible Member and Distractor Tip

In some examples, the needle device is constructed or otherwise manufactured to include a resilient flexible member with a distractor tip, that is, a device with integrated and/or assembled components. In such examples, the user may not need to assemble the distraction device 100 with the needle device 102 (as was the case of the example illustrated in FIG. 1).

Therefore, the needle device for insertion into tissue can include a body, a resilient flexible member, and a needle. The body can facilitate ease of handling of the needle. The body can also serve as a base to which other components are mounted.

The resiliently flexible member extending from the body of the needle device, wherein the resiliently flexible member includes a distractor tip. The resilient flexible member can be the same or similar to the examples described above, but extends from the body of the needle device instead of a separate distractor device body.

The needle device also includes a needle with a needle tip. The needle may be fixed relative to the body of the needle device. In other examples, the needle may be selectively moveable relative to the body. This can include selectively sliding the needle into the body so that the needle is retracted. Selective retraction can be used to prevent, or reduce, the sharps hazard of a needle after use.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The various embodiments described above can be combined to provide further embodiments. These and other changes can be made to the embodiments in light of the above-detailed description. In general, in the following claims, the terms used should not be construed to limit the claims to the specific embodiments disclosed in the specification, but should be construed to include all possible embodiments along with the full scope of equivalents to which such claims are entitled.

1. A distraction device configured to be used with a needle device for insertion into tissue, comprising:
a body attached to the needle device; and
a member extending from the body, the member comprising a distractor tip proximal to a needle tip of the needle device;
wherein in use the distractor tip is configured to contact a tissue surface before and during insertion of the needle tip into the tissue surface.

2. The distraction device of claim 1, wherein the body is removably attached to the needle device.

3. The distraction device of claim 1, wherein the body comprises an adapter to interface the body with the needle device.

4. The distraction device of claim 3, wherein the adapter comprises a slotted tube to slidingly receive the needle device.

5. The distraction device of claim 1, wherein the member is removably attached to the body.

6. The distraction device of claim 1, wherein the member is resiliently flexible such that the member is configured to bend when the distractor tip is in contact with the tissue surface during insertion of the needle tip into the tissue surface.

7. The distraction device of claim 6, wherein the resiliently flexible member is configured to apply pressure on the tissue surface without causing penetration.

8. The distraction device of claim 6, wherein the resiliently flexible member allows the distractor tip to remain at a same location on the tissue surface during insertion of the needle tip.

9. The distraction device of claim 1, wherein the distractor tip is located ahead of the needle tip of the needle device.

10. The distraction device of claim 1, wherein the distractor tip is located adjacent to the needle tip of the needle device.

11. The distraction device of claim 1, further comprising a stabilizer grip to stabilize the distraction device before and during insertion of the needle tip into the tissue surface.

12. The distraction device of claim 11, wherein the stabiliser stabilizer grip comprises a pair of resilient flaps that are selectively attached to the needle device.

13. The distraction device of claim 12, wherein the pair of resilient flaps fold towards the body such that a digits of an operator of the needle device can squeeze the stabilizer grips with part of the body between.

14. A needle device for insertion into tissue, comprising:
a body;
a resiliently flexible member extending from the body of the needle device, wherein the resiliently flexible member includes a distractor tip; and
a needle with a needle tip,
wherein the distractor tip is proximal to the needle tip, wherein in use the distractor tip is configured to contact a tissue surface of a patient before and during insertion of the needle tip into the tissue surface.

15. The needle device of claim 14, wherein the resiliently flexible member is configured to bend during insertion of the needle tip into the tissue surface.

16. The needle device of claim 14, wherein the resiliently flexible member is configured to apply pressure to the tissue surface without causing penetration.

17. The needle device of claim 14, wherein the resiliently flexible member allows the distractor tip to remain at a same location on the tissue surface during insertion of the needle tip.

18. The needle device according to claim 14, wherein the distractor tip is located ahead of the needle tip.

19. The needle device according to claim 14, wherein the distractor tip is located adjacent to the needle tip of the needle device.
